(19) United States
(12) Patent Application Publication
(10) Pub. No.: US 2007/0173700 A1
(43) Pub. Date: Jul. 26, 2007

(54) Disease risk information display device and program

(76) Inventors: Akinori Ishihara, Chiba (JP); Issei Tamada, Chiba (JP); Junichiro Miura, Chiba (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 11/348,404

(22) Filed: Feb. 7, 2006

(30) Foreign Application Priority Data
Jan. 25, 2006 (JP) 2006-016807

(51) Int. Cl. A61B 5/00; G06T 11/20

(52) U.S. Cl. 600/300; 128/920; 345/440

(57) ABSTRACT

A disease risk information display device and program, for enabling straightforward understanding of which degree of risk there is to what kind of disease, is provided with an analysis processing section 18 for carrying out singular value decomposition taking medical data for a plurality of individuals as input data and outputting a set of singular values characterizing the physical condition of each individual, and a diagram production section 19 for determining coordinates representing each individual in two-dimensional coordinate space based on the singular values, selecting a plurality of reference points in two-dimensional coordinate space, calculating the possibility of individuals represented by coordinates of each reference point contracting a certain disease based on statistical data, determining coordinates in a height direction of the reference points based on calculated possibility, and producing three-dimensional diagrams. When input of singular values for subjects is received, two-dimensional coordinates representing the subjects are determined based on the singular values, and points corresponding to the subjects are arranged in a three-dimensional diagram.

### BACKGROUND OF THE INVENTION

1. Field of the Invention

The present invention relates to a disease risk information display device and program.

2. Description of Related Art

Data analysis techniques such as multivariate analysis are widely utilized in the field of medical care. For example, various research is taking place into methods for predicting the extent of risk certain patients are exposed to with respect to certain diseases by collectively analyzing various clinical data and lifestyle habit data etc.

For example, in JP-A-2004-518463, a method is disclosed for classifying patients into a plurality of sets according to points for disease conditions and risk conditions relating to selected biological diseases. Here, points are assigned to results of medical screening including genetic screening and risk factors and weightings are assigned according to the extent of correlation in deciding disease conditions and risk, Points are then automatically assigned for the medical screening results and clinical history of individual patients within a set in accordance with predetermined characteristics.

Various types of data are employed in this data analysis. For example, proteomics data expressing the expression level of each various protein appearing every patient etc. is useful in predicting whether or not a patient may have contracted a certain disease.

Singular value decomposition is well-known as a method of arranging each individual in space with a low number of dimensions, i.e. as a method of expressing characteristics of these individuals using fewer parameters in the event that a plurality of items of data (variables) are provided for a certain patient (sample).

According to singular value decomposition, at the same time as it being possible to arrange each sample in space of a low number of dimensions, it is also possible to arrange each variable using analysis in the same space. This makes it possible to see the relationship between samples and variables.

For example, when proteomics data is analyzed using singular value decomposition, it is possible to know the protein distribution at the same time as the sample distribution in the same two-dimensional space.

The results of the singular value decomposition are typically visualized using a two-dimensional or three-dimensional scatter diagram. However, scatter diagrams are not easy particularly for a patient to understand which degree of risk they themselves have to what kind of disease.

It is therefore advantageous for the present invention to provide a disease risk information display device and program where it is easy to understand which degree of risk there is to what kind of disease.

### SUMMARY OF THE INVENTION

The disease risk information display device of the present invention comprises an analysis processor for carrying out analysis processing taking medical data for a plurality of individuals as input data and outputting a set of parameters characterizing the physical condition of each individual, and a diagram production section for determining coordinates representing each individual in two-dimensional coordinate space based on the set of parameters, selecting a plurality of reference points in two-dimensional coordinate space, calculating the possibility of individuals represented by coordinates of each reference point contracting a certain disease based on statistical data, determining coordinates in a height direction of the reference points based on calculated possibility, and producing three-dimensional diagrams. When input for values for sets of parameters for subjects are received, the diagram production section determines two-dimensional coordinates representing the subjects based on the values for the sets of parameters, and arranges points corresponding to the subjects on the three-dimensional diagram.

As a result, it is possible to visually comprehend the extent to which it is possible that a subject may have contracted a certain disease.

It is also preferable for the analysis processor to analyze the medical data using singular value decomposition and output sets of singular values characterizing the physical condition of each individual, and for the diagram production section to select two singular values from the set of the singular values, and determine coordinates representing each individual in two-dimensional coordinate space taking the respective singular values as coordinates X, Y.

Further, it is also possible to utilize, for example, data representing expression level of each type of protein occurring in an individual as medical data. Pathological data, clinical data such as blood pressure, height, weight, and blood-sugar level, and lifestyle habit data such as smoking, drinking, diet, and sleep may also be utilized for each individual.

Further, the statistical data may also be the “probability of an individual contracting a certain disease” expressed using coordinates of a plurality of points in the vicinity of each reference point.

Further, it is preferable for the diagram production section to correct coordinates in a height direction of the plurality of reference points according to the type of disease.

As a result, it is possible, for example, to represent more serious illnesses as being higher in a three-dimensional diagram, It is therefore possible for a subject to visually comprehend the magnitude of risk associated with a disease.

For example, the diagram production section is capable of correcting coordinates in a height direction of the plurality of reference points by dividing into survival rates for after five years for each disease. In addition, it is possible to employ an index representing risk according to type of disease.

Further, the diagram production section determines coordinates in a height direction of the plurality of reference points for a plurality of diseases, and takes the highest of coordinates determined in a height direction as coordinates in a height direction of the respective reference points for each reference point.

As a result, it is possible to represent the possibility of contraction relating to a plurality of diseases using a single three-dimensional diagram. This makes it possible to visually comprehend the possibility of contraction of complications for a plurality of diseases.

Further, it is possible for the three-dimensional diagram to be a solid figure resembling a mountain.

In this way, it is possible to represent conditions of a disease of a subject in the way a mountaineer views a mountain representing a disease and this is easy to understand for a subject.

A program of the present invention causes a computer to function as data analysis processor means for carrying out analysis processing taking medical data for a plurality of individuals as input data and outputting a set of parameters characterizing the physical condition of each individual, and three-dimensional diagram production means for determining coordinates representing each individual in two-dimensional coordinate space based on the set of parameters, selecting a plurality of reference points in two-dimensional coordinate space, calculating the possibility of individuals represented by coordinates of each reference point contracting a certain disease based on statistical data, determining coordinates in a height direction of the reference points based on calculated possibility, and producing three-dimensional diagrams. When input for values for sets of parameters for subjects are received, the three-dimensional diagram production means determines two-dimensional coordinates representing the subjects based on the values for the sets of parameters, and arranges points corresponding to the subjects on the three-dimensional diagram.

As a result, it is possible to visually comprehend the extent to which it is possible that a subject themselves may have contracted a certain disease.

### BRIEF DESCRIPTION OF DRAWINGS

FIG. 1 is a block view showing a configuration for a disease risk information display device of a first embodiment of the present invention.

FIG. 2 is a flowchart of three-dimensional diagram production processing performed by a diagram production section of the first embodiment.

FIG. 3. is a view representing the relationship between the reference points and points representing each individual in two-dimensional coordinate space taking singular value 1 as an X-coordinate and singular value 2 as a Y-coordinate.

FIG. 4 is a view illustrating a method for calculating the possibility of contracting a disease occurring at a reference point.

FIG. 5A is a view representing a disease risk map in two-dimensional space. FIG. 5B is a view in vertical cross-section (a cross-sectional view along line C-C′ in FIG. 5A) of the disease risk map in an X-Y plane.

FIG. 6A is a view showing distribution of points representing each individual in two-dimensional coordinate space, and FIG. 6B is a view showing a disease risk map corresponding to FIG. 6A.

FIG. 7A is a further view showing distribution of points representing each individual in two-dimensional coordinate space, and FIG. 7B is a view showing a disease risk map corresponding to FIG. 7A.

FIG. 8A is a view representing a disease risk map for disease A and disease B in two-dimensional space, and FIG. 8B is a view of a disease risk map for disease A and disease B cut-away as a vertical cross-section (along line C-C′ in the drawing) in an X-Y plane.

FIG. 9A is a view where disease risk maps for disease A and disease B shown in FIG. BA are superimposed, and FIG. 9B is a view where disease risk maps for disease A and disease B shown in FIG. 8B are superimposed.

FIG. 10 is a view showing an example of a three-dimensional diagram where points corresponding to subjects are arranged on a disease risk map relating to a plurality of diseases.

FIG. 11 is a further view showing an example of a three-dimensional diagram where points corresponding to subjects are arranged on a disease risk map relating to a plurality of diseases.

FIG. 12 is an example of a three-dimensional diagram where points corresponding to subjects are arranged on a disease risk map relating to a plurality of diseases shown as a two-dimensional diagram.

FIG. 13 is a view showing a two-dimensional scatter diagram of singular values of a comparative example.

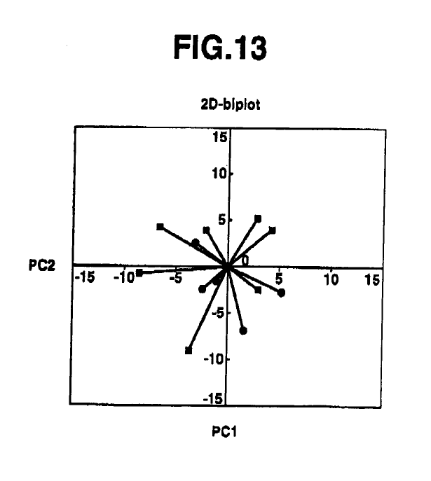

### DESCRIPTION OF THE PREFERRED EMBODIMENTS

The following is a description with reference to the drawings of an embodiment of the present invention.

### First Embodiment

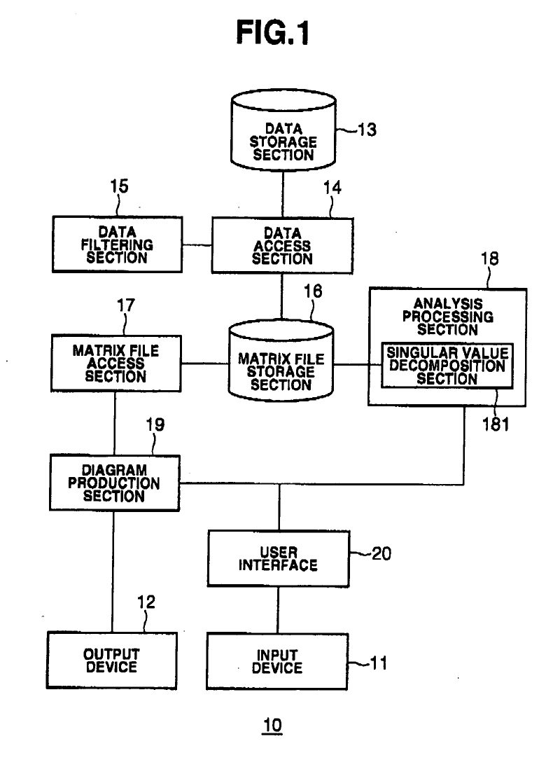

FIG. 1 is a block view showing a configuration for a disease risk information display device 10 of a first embodiment of the present invention. As shown in FIG. 1, the disease risk information display device 10 is comprised of input device 11, output device 12, a data storage section 13, a data access section 14, a data filtering section 15, a matrix file storage section 16, a matrix file access section 17, an analysis processing section 18, a diagram production section 19, and a user interface 20. The analysis processing section 18 is provided with a singular value decomposition section 181.

The disease risk information display device 10 may be a predetermined program executed, for example, on a general purpose personal computer. The data access section 14, data filtering section 15, matrix file access section 17, analysis processing section 18, diagram production section 19 and user interface 20 represent modules for operations carried out by a processor of a computer in accordance with a program and are actually integrated in the configuration for a processor of the disease risk information display device 10.

Data storage section 13 and matrix file storage section 16 are storage devices such as a hard disc etc. of the disease risk information display device 10.

The input device 11 is constituted by input means such as, for example, a keyboard, mouse, or touch-sensitive panel etc. and is used for the user to designate processing at the disease risk information display device 10 and assign parameters.

Output device 12 is a display device or printer etc., and outputs diagram date etc.

Medical data constituting input data for data analysis processing is stored in the data storage section 13. Proteomics data for a plurality of individuals is included in this data.

Proteomics data is data quantitatively expressing how much of what kind of protein appears for a certain individual. Specifically, an antigen-antibody reaction is carried out using a monoclonal antibody identifying each protein (antigen) including qualitative variance such as modification after translation etc. specifically and a sample such as a serum for an individual, and strength of the reaction is quantified. This value shows the expression level of antigenic proteins for this individual,

Typically, in the event that an individual has contracted a certain disease or is highly likely to contract a disease in the future, it is considered to change the expression level of protein closely related to this disease. The proteomics data is therefore effective in predicting the magnitude (possibility of contraction) of risk with. respect to various diseases for an individual.

Proteomics data obtained is then stored in the matrix file storage section 16 in a matrix file format formed so as to combine the individuals (samples) 1 to n and proteins 1 to m shown in table 1. Further, the matrix file is stored in the data storage section 13 as a binary file.

TABLE 1

| | Sample 1 | Sample 2 | . . . | Sample n |
|---|---|---|---|---|
| Protein 1 | 0.1 | 0.2 | . . . | 0.1 |
| Protein 2 | 0.5 | 0.5 | . . . | 0.6 |
| . | . | . | . . . | . |
| . | . | . | | . |
| . | . | . | | . |
| Protein m | 0.3 | 0.7 | . . . | 0.1 |

Moreover, data showing whether or not individuals are suffering from diseases is also stored in the data storage section 13 for various types of diseases.

In addition, pathological data, clinical data such as blood pressure, height, weight, and blood-sugar level, and lifestyle habit data such as smoking, drinking, diet, and sleep can be stored for each individual. Each module of the disease risk information display device 10 accesses a database within the data storage section 13 via the data access section 14. At this time, it is possible to extract only data fulfilling predetermined conditions via the data filtering section 15. Further, each module of the disease risk information display device 10 accesses matrix files within the matrix file storage section 16 via the matrix file access section 17.

A description is now given of the operation of the disease risk information display device 10.

First, a description is given of data analysis processing by the analysis processing section 15.

When the user provides a data analysis processing instruction via the input device 11, the user interface 20 provides a data analysis processing instruction to the analysis processing section 18. Here, singular value decomposition is used as the data analysis method. Further, the proteomics data described above is used as input data.

First, analysis processing section 18 acquires proteomics data from the matrix file storage section 16 via the matrix file access section 17.

The singular value decomposition section 181 of the analysis processing section 18 executes data analysis using singular value decomposition taking acquired proteomics data as input data.

Specifically, the singular value decomposition section 181 carries out matrix computations in such a manner that when matrix (m×n) for proteomics data is taken to be A, a matrix (m×r) possessing a normalized orthogonal vector as a column vector is taken to be U, a diagonal matrix (r×r) possessing λ1, . . . λr (where r is the order) as diagonal elements is taken to be W, and a matrix (r×n) possessing a normalized orthogonal vector as a column vector is taken to be V^{T}, the matrix U, V^{T} for which the relationship of equation (1) holds true is computed.
A=U·W·V^{T} (1)

An example of the matrix U is shown in table 2 and an example of the matrix V^{T} is shown in table 3. The matrix U is a matrix comprised of singular values 1 to r for each protein 1 to m, and the matrix V^{T} is a matrix comprised of singular values 1 to r for each individual (sample). At the matrix V^{T}, the singular values 1 to r of each individual (sample) constitute sets of parameters characterizing the physical condition of each individual. Here, “physical condition” refers mainly to the magnitude of risk of an individual with respect to various diseases, i.e. the possibility of an individual having contracted a certain disease or the possibility of the disease being contracted in the future. Further, the singular values 1 to r of the proteins 1 to m constitute sets of parameters characterizing the relationship between each protein and various diseases.

TABLE 2

| | singular | singular | | singular |
|---|---|---|---|---|
| | value 1 | value 2 | . . . | value r |
| Protein 1 | 1 | 10 | . . . | 4 |
| Protein 2 | 3 | 2 | . . . | 6 |
| . | . | . | . . . | . |
| . | . | . | | . |
| . | . | . | | . |
| Protein m | 5 | 3 | . . . | 7 |

TABLE 3

| | sample 1 | sample 2 | . . . | sample n |
|---|---|---|---|---|
| singular | 7 | 3 | . . . | 3 |
| value 1 | | | | |
| singular | 4 | 1 | . . . | 2 |
| value 2 | | | | |
| . | . | . | . . . | . |
| . | . | . | | . |
| . | . | . | | . |
| singular | 5 | 6 | . . . | 3 |
| value r | | | | |

The results of analysis processing by the analysis processing section 18 are stored in the matrix file storage section 16 as the matrix files shown in table 2 and table 3, Further, the data access section 14 acquires and stores in the data storage section 13 as binary files matrix files for singular value decomposition results from the matrix file storage section 16.

Next, a description is given of three-dimensional diagram production processing by the diagram production section 19.

When the user provides a three-dimensional diagram production processing instruction via the input device 11, the user interface 20 provides a three-dimensional diagram production processing instruction to the diagram production section 19.

The diagram production section 19 produces three-dimensional diagrams using the results of analysis processing by the analysis processing section 18.

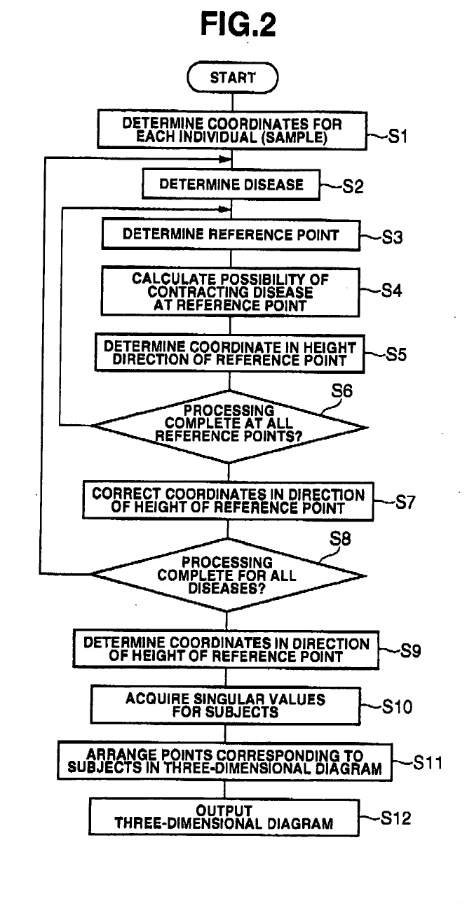

FIG. 2 is a flowchart of three-dimensional diagram production processing performed by the diagram production section 19.

First, the diagram production section 19 decides coordinates representing each individual in two-dimensional coordinate space (step S1).

Specifically, first, a matrix file V^{T} constituted by singular values 1 to r for a plurality of individuals (samples) is acquired from the matrix file storage section 16 via the matrix file access section 17, and two singular values (for example, singular value 1 and singular value 2) are selected from the singular values 1 to r. Singular value 1 is then decided as an X-coordinate and singular value 2 is decided as a Y-coordinate.

It is possible for the used analysis results matrix file and selected singular values to be designated by the user from the input device 11 via the user interface 20.

Next, the diagram production section 19 decides upon a single disease to be taken note of (step S2). Specifically, this is carried out by selecting one from one or more types of disease specified by the user via the input device 11.

Next, the diagram production section 19 decides upon one point (X1, Y1) in an X-Y plane as a reference point (step S3). Here, a “reference point” represents a point deciding a z coordinate (coordinate in a height direction) for producing a three-dimensional diagram.

Specifically, the diagram production section 19 determines N reference points in such a manner as to give uniform distribution in an X-Y plane based on the total number of reference points designated by the user via the input device 11, and one point is selected from these points.

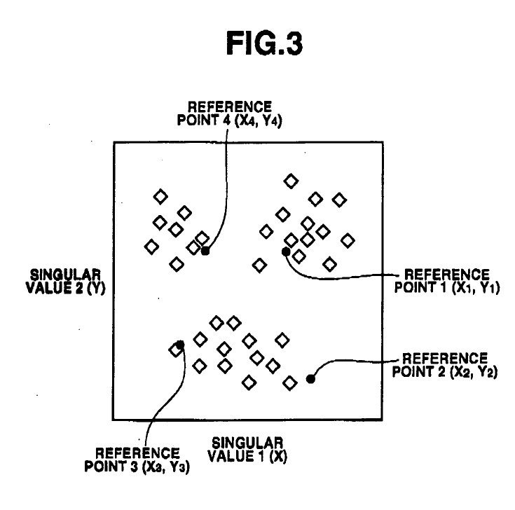

FIG. 3 is a view representing the relationship between the reference points and points representing each individual in two-dimensional coordinate space taking singular value 1 as an X-coordinate and singular value 2 as a Y-coordinate. Here, four reference points are shown. In the drawings, each reference point is shown by a black circle, and points representing each individual are shown by a square.

Next, the diagram production section 19 calculates the possibility of an individual represented by the coordinate of reference point (X1, Y1) contracting the disease (taken here to be “disease A”) determined in step S2 based on statistical data (step S4).

Statistical data is constituted by the probability of individuals expressed by a plurality of points in the vicinity of the reference point (X1, Y1) having contracted a certain disease.

First, the diagram production section 19 counts a number N1 of points representing individuals existing within a circle of radius R taking point (X1, Y1) as center in the vicinity of the reference point (X1, Y1). The radius R can be designated by the user from the input device 11 via the user interface 20.

Next, the diagram production section 19 counts a number N2 of individuals actually suffering from disease A of the individuals expressed by the points existing within a circle of radius R taking a point (X1, Y1) as center. The number of individuals suffering from disease A is calculated, for example, by acquiring data indicating whether or not each individual is afflicted with disease A from the data storage section 13 and then performing calculations.

Next, the diagram production section 19 calculates the ratio of N2 with respect to N1. The calculated ratio expresses the possibility (disease risk value) of having contracted disease A at reference point (X1, Y1).

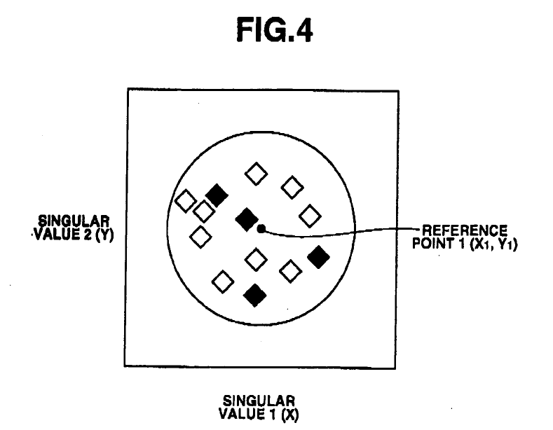

A detailed description is now given of a method of calculating disease risk value occurring at reference point (X1, Y1) using FIG. 4. In the drawings, squares that are not filled in are points representing individuals that have contracted disease A, and squares that are filled in are points representing individuals that have not contracted disease A. In the example in FIG. 4, N1 is 13 and N2 is 9. Disease risk value risk A for disease A then becomes 69.2%, as shown in equation (2).
riskA= 9/13*100=69.2(%) (2)

Next, diagram production section 19 substitutes disease risk value risk A occurring at reference point (X1, Y1) calculated in step S4 at a coordinate (z coordinate) in a height direction of reference point (X1, Y1) and takes the disease risk value risk A occurring at reference point (X1, Y1) calculated in step S4 as a z coordinate for reference point (X1, Y1) (step S5). An initial value for the coordinate (z coordinate) in the height direction is 0, and the initial value is adopted in the event that a point representing an individual in the vicinity does not exist (N1=0).

The method of calculating the disease risk value A is by no means limited to the above, and, for example, calculations employing methods such as logistic regression analysis is also possible.

Next, the diagram production section 19 determines whether or not step S3 to step S5 is complete for all reference points determined based on user designations (step S6). If processing for all reference points is not complete, step S3 is returned to, the next reference point is determined, and processing is repeated.

When processing is complete for all of the reference points, z coordinates (disease risk values) for each reference point have been determined. It is therefore possible to arrange each reference point in three-dimensional coordinate space. A distribution map in three-dimensional coordinate space obtained in this manner is referred to as a disease risk map for disease A.

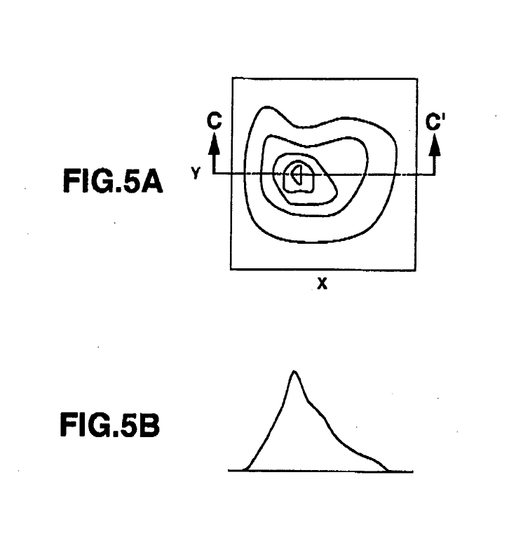

FIG. 5A is a view representing a disease risk map in two-dimensional space. FIG. 5B is a view in vertical cross-section (a cross-sectional view along line C-C′ in FIG. 5A) of the disease risk map in an X-Y plane.

Each curve shown in FIG. 5A is a line linking points where disease risk value (z coordinate) is equal. As shown in the drawing, when points where the disease risk value is equal are linked, a view that appears as contour lines on a map is obtained.

A description is now given of the reason disease risk value distribution is a contour line distribution. As described above, disease risk values for each reference point are calculated using points positioned in the vicinity of each respective reference point (within a circle of radius R). A large number of the points used in calculation of disease risk values for two arbitrary reference points at close positions in coordinate space are therefore common. As a result, disease risk values for reference points positioned closely in coordinate space are close values.

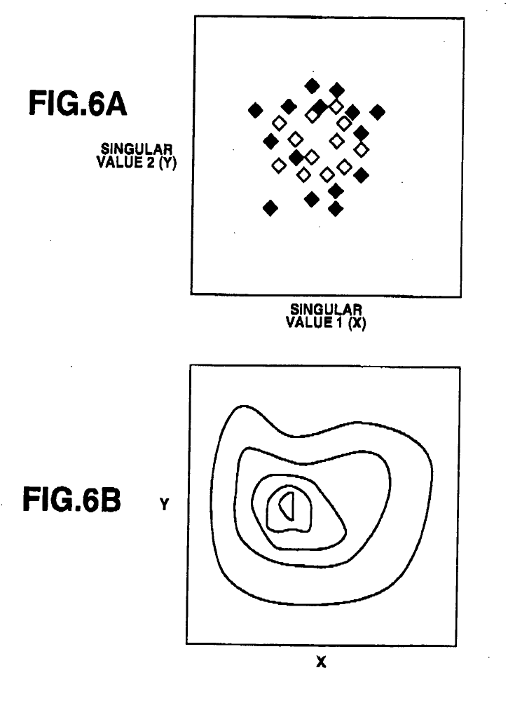

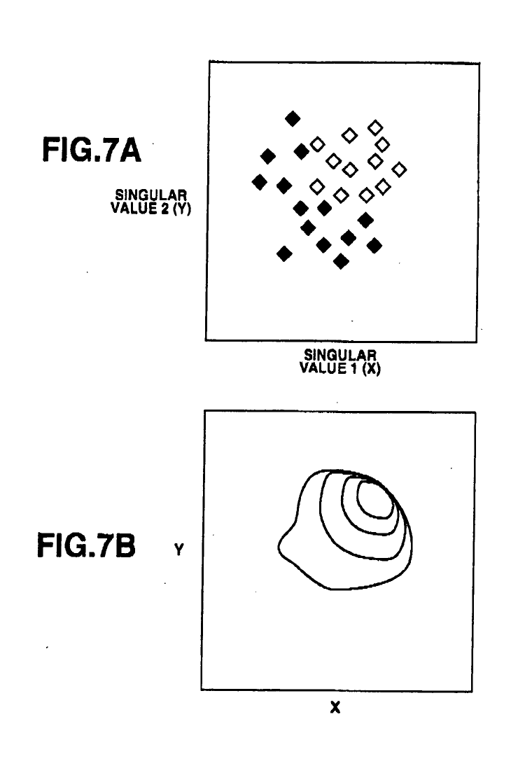

Further, the disease risk map is of a shape corresponding to the distribution of disease risk values. FIG. 6 and FIG. 7 are views showing the relationship between distribution of points expressing each individual and disease risk maps in two-dimensional coordinate space. In the drawings, squares that are not filled in are points representing individuals that have contracted disease A, and squares that are filled in are points representing individuals that have not contracted disease A.

In the example shown in FIG. 6A, points representing individuals that have contracted disease A are concentrated at a central section of two-dimensional coordinate space.

In this event, the disease risk map is in the shape of a mountain where the altitude (z coordinate) of a central section is high, as shown in FIG. 6B.

Further, in the example shown in FIG. 7A, points representing individuals that have contracted disease A are concentrated at the upper right of the drawing.

In this event, the disease risk map is in the shape of a mountain where the right end is as a cliff, as shown in FIG. 7B.

When it is determined that processing is complete for all reference points in step S6, the diagram production section 19 corrects the disease risk values (z coordinate) occurring at all of the reference points according to disease type (step S7). Here, the diagram production section 19 corrects by dividing the z coordinates for all of the reference points by the survival rate for after five years for the disease A.

For example, when the survival rate after five years for disease A is taken to be 30%, a disease risk value risk′ A for after correction of disease A is obtained from equation (3),
risk′A=risk A/0.3 (3)

As a result of this correction, diseases where the five year survival rate is low, i.e. diseases where the disease itself is highly dangerous, have a higher z coordinate after correction. The five year survival rate for disease A can be designated by the user from the input device 11 via the user interface 20. The numerical value used in correction is by no means limited to five year survival rate and that expressing risk according to type of disease is also possible.

Next, the diagram production section 19 determines whether or not step S2 to step S7 is complete for all diseases designated by the user (step S8). If processing for all of the diseases is not complete, step S2 is returned to, the next disease is determined, and processing is repeated.

If it is determined that processing is complete for all diseases in step S8, the diagram production section 19 again determines the disease risk values (z coordinate) occurring at all of the reference points (step S9).

Specifically, the diagram production section 19 selects the highest from the z-coordinates of each reference point determined for a plurality of diseases to be taken as z-coordinates of respective reference points.

When step S9 is complete z coordinates (disease risk values) are determined for each reference point taking into consideration disease risk values for all of the diseases. When each reference point is arranged in three-dimensional coordinate space, a disease risk map relating to a plurality of diseases is obtained.

The disease risk map relating to a plurality of diseases is the disease risk maps for the respective diseases superimposed.

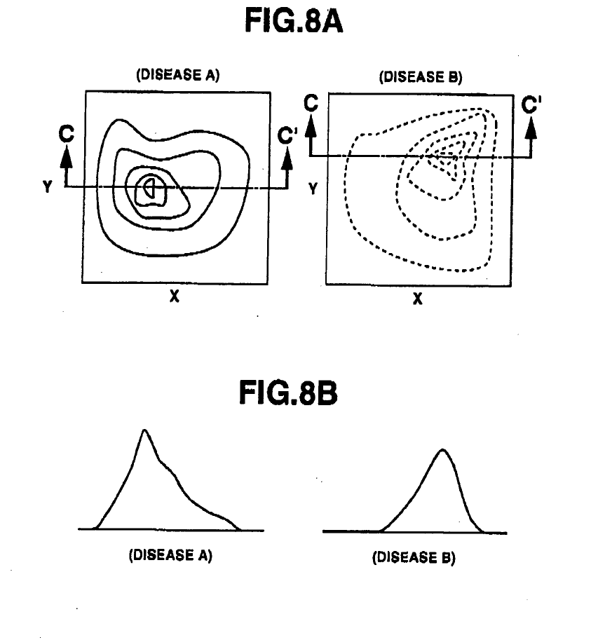

FIG. 8A is a view where a plurality of disease risk maps for disease A and disease B are represented in two-dimensional space, as with FIG. 5A, and FIG. 8B is a view cut-away in vertical cross-section (a cross-sectional view along line C-C′ in FIG. 8A) in respective X-Y planes for the disease risk maps for disease A and disease B, as in FIG. 5B.

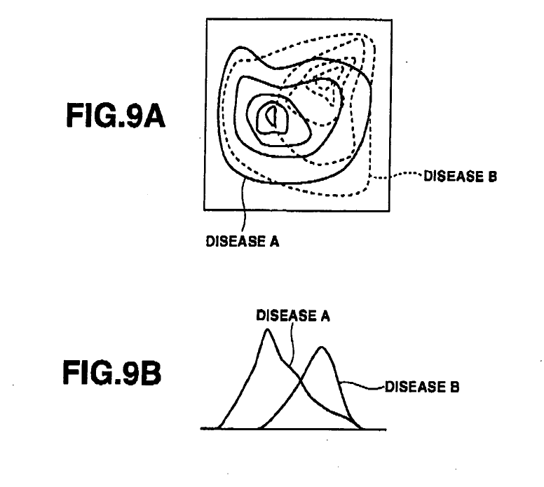

FIG. 9A is a view where disease risk maps for disease A and disease B shown in FIG. 8A are superimposed, and FIG. 9B is a view where disease risk maps for disease A and disease B shown in FIG. 8B are superimposed.

As described above, the diagram production section 19 makes a three-dimensional diagram taking the z coordinate values for the diseases having the largest z coordinates for each reference point as z coordinates for the reference points. Further, because the z coordinates of the reference points are corrected using five year survival rates of the respective diseases, even if the disease risk value is the same, disease risk maps for diseases that are more dangerous have larger z coordinates.

As a result, when, for example, five year survival rate for disease A is taken to be 30%, five year survival rate for disease B is taken to be 60%, the disease risk value (z coordinate) before correction for disease A occurring at reference point (X1, Y1) is taken to be 0.3, and the disease risk value (z coordinate) before correction for disease B is taken to be 0.4, the disease risk for disease B is higher for disease risk before correction but disease risk (z coordinate) after correction at point (X1, Y1) becomes 0.3/0.3=1 for disease A and becomes 0.4/0.66 for disease B, so that disease A becomes larger. A value for disease A is therefore selected as the z coordinate for reference point (X1, Y1).

As shown in FIG. 9A and FIG. 9B, part of the mountains for disease risk for disease A and disease B overlap. Typically, correlation is stronger when the overlapping portions of the mountains for disease A and disease B are greater, which may more easily lead to complications.

Next, the diagram production section 19 acquires values for the singular value 1 and the singular value 2 for subjects designated by the user using the input device 11 via the user interface 20 (step S10).

After this, the diagram production section 19 takes acquired singular value 1 and singular value 2 for a subject as an X-coordinate and Y-coordinate, respectively, and arranges points corresponding to the subjects on a disease risk map relating to a plurality of diseases obtained in step S9 (step S11).

Next, the diagram production section 19 outputs the three-dimensional diagram arranged with points corresponding to subjects on a disease risk map relating to a plurality of diseases obtained in step S11 at the output device 12 (step S12).

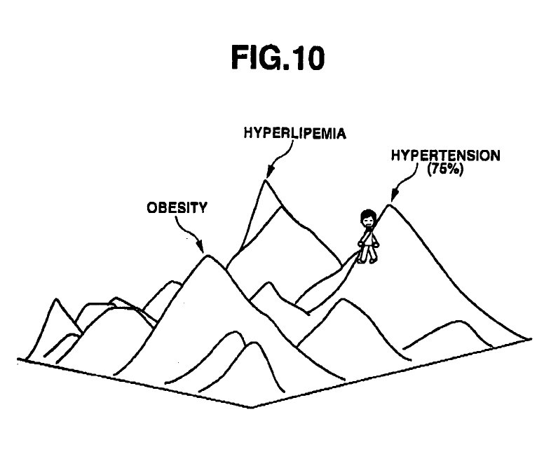

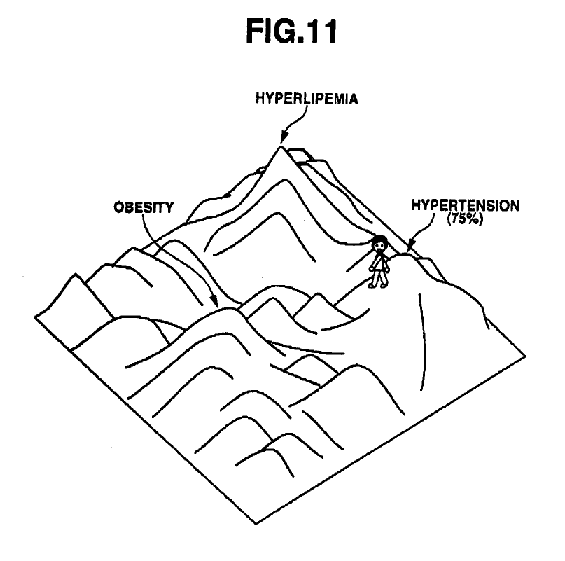

FIG. 10 and FIG. 11 are three-dimensional diagrams of the arrangement of points corresponding to subjects on a disease map relating to a plurality of diseases outputted in step S12 viewed from different angles.

As shown in the drawing, the three-dimensional diagram is a solid figure resembling a mountain. The respective mountains are mountains corresponding to disease risk maps for certain respective diseases. Here, mountains are displayed corresponding to risk maps for three types of diseases of hypertension, hyperlipemia, and obesity. When the possibility of a subject contracting hypertension is taken to be 75%, as shown in the drawing, a figure representing a subject is arranged in the vicinity of the 75% of the way up to the top of the mountain for hypertension of the disease risk map, and it is possible for a subject to visually comprehend the extent of risk with respect to particular diseases.

In the event that a figure representing a subject is arranged at a position where, for example, ridge lines for a mountain for hypertension and for a mountain for hyperlipemia overlap, it is possible for a subject to visually comprehend risk with respect to both the diseases of hypertension and hyperlipemia.

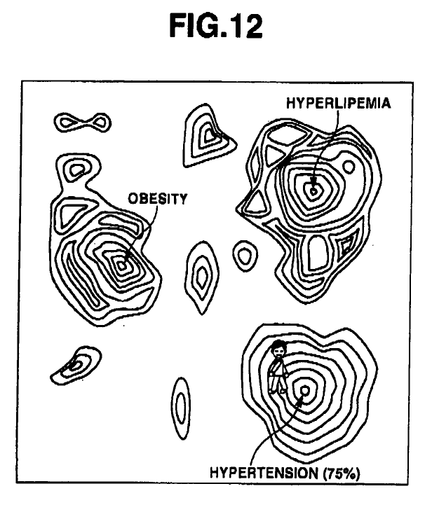

Further, FIG. 12 is an example of a three-dimensional diagram where points corresponding to subjects are arranged on a disease risk map relating to a plurality of diseases shown as a two-dimensional shape. The diagram production section 19 is also capable of outputting this kind of two-dimensional diagram.

The diagram production section 19 is capable of arranging points representing each protein on a disease risk map relating to a plurality of diseases taking singular value 1 as an X-coordinate and singular value 2 as a Y-coordinate for each protein shown in table 2. As a result, it is possible to comprehend the correlation of each protein and each disease in a three-dimensional diagram.

FIG. 11 is a view showing a two-dimensional scatter diagram of singular values of a comparative example. Even if points representing subjects are arranged in two dimensional space as shown in FIG. 11, it is difficult to understand specifically to what extent a subject is likely to contract which disease.

A three-dimensional diagram produced by the disease risk information display device 10 of the first embodiment has the advantage compared to the typical two-dimensional scatter diagram of singular values shown in FIG. 11 in that it is easy for a subject to understand the possibility of having contracted a certain disease and the possibility of complications with regards to other strongly related diseases.

What is claimed is:

1. A disease risk information display device comprising:
an analysis processor for carrying out analysis processing taking medical data for a plurality of individuals as input data and outputting a set of parameters characterizing the physical condition of each individual; and
a diagram production section for determining coordinates representing each individual in two-dimensional coordinate space based on the set of parameters, selecting a plurality of reference points in two-dimensional coordinate space, calculating the possibility of individuals represented by coordinates of each reference point contracting a certain disease based on statistical data, determining. coordinates in a height direction of the reference points based on calculated possibility, and producing three-dimensional diagrams,
wherein when input for values for sets of parameters for subjects are received, the diagram production section determines two-dimensional coordinates representing the subjects based on the values for the sets of parameters, and arranges points corresponding to the subjects on the three-dimensional diagram.

2. The disease risk information display device according to claim 1, wherein the analysis processor analyzes the medical data using singular value decomposition and outputs sets of singular values characterizing the physical condition of each individual, and
the diagram production section selects two singular values from the set of singular values, and determines coordinates representing each individual in two-dimensional coordinate space taking the respective singular values as coordinates X, Y.

3. The disease risk information display device according to claim 2, wherein the medical data is data representing expression level of each type of protein occurring in an individual.

4. The disease risk information display device according to claim 1, wherein the statistical data is constituted by probability of an individual contracting a certain disease expressed by coordinates of a plurality of points in the vicinity of each reference point.

5. The disease risk information display device according to claim 1, wherein the diagram production section corrects coordinates in a height direction of the plurality of reference points according to the type of disease.

6. The disease risk information display device according to claim 5, wherein the diagram production section corrects coordinates in a height direction of the plurality of reference points by dividing into survival rates for after five years for each disease.

7. The disease risk information display device according to claim 1, wherein the diagram production section determines coordinates in a height direction of the plurality of reference points for a plurality of diseases, and takes the highest of coordinates determined in a height direction as coordinates for the height direction of the respective reference points for each reference point.

8. The disease risk information display device according to claim 1, wherein the three-dimensional diagram is a solid figure resembling a mountain.

9. A program causing a computer to function as:
data analysis processor means for carrying out analysis processing taking medical data for a plurality of individuals as input data and outputting a set of parameters characterizing the physical condition of each individual; and
three-dimensional diagram production means for determining coordinates representing each individual in two-dimensional coordinate space based on the set of parameters, selecting a plurality of reference points in two-dimensional coordinate space, calculating the possibility of individuals represented by coordinates of each reference point contracting a certain disease based on statistical data, determining coordinates in a height direction of the reference points based on calculated possibility, and producing three-dimensional diagrams,
wherein when input for values for sets of parameters for subjects are received, the three-dimensional diagram production means determines two-dimensional coordinates representing the subjects based on the values for the sets of parameters, and arranges points corresponding to the subjects on the three-dimensional diagram.
